(19) United States
(12) Patent Application Publication
(10) Pub. No.: US 2012/0142929 A1
(43) Pub. Date: Jun. 7, 2012

(54) PROCESS FOR PREPARING SULFONYL QUINOLINES

(75) Inventors: Nitinchandra D. Patel, Danbury, CT (US); Chris H. Senanayake, Brookfield, CT (US); Xudong Wei, Ridgefield, CT (US); Nathan K. Yee, Danbury, CT (US)

(73) Assignee: BOEHRINGER INGELHEIM INTERNATIONAL GMBH, Ingelheim am Rhein (DE)

(21) Appl. No.: 13/256,752

(22) PCT Filed: Mar. 18, 2010
(86) PCT No.: PCT/US2010/027747

§ 371 Date: Jan. 5, 2012

Related U.S. Application Data
(60) Provisional application No. 61/161,452, filed on Mar. 19, 2009.

(51) Int. Cl. C07D 411/04

(52) U.S. Cl. 546/153

(57) ABSTRACT

Disclosed are highly convergent processes for preparing compounds of formula (I), which compounds are useful as intermediates in the preparation of potent active agents for the treatment of hepatitis C virus (HCV) infection.

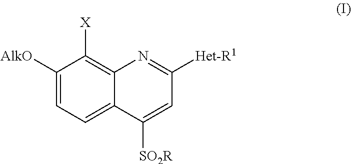

### BACKGROUND OF THE INVENTION

1. Technical Field

The application includes a description of improved processes for the preparation of substituted 4-sulfonyl quinolines which are useful as intermediates in the preparation of agents for the treatment of hepatitis C viral (HCV) infections.

2. Background Information

4-Sulfonyl substituted quinolines which are preparable according to the methods described herein have been found to be useful as intermediates in the preparation of certain anti-HCV agents. Examples of such anti-HCV agents are described, e.g., in U.S. Patent Application Publication Nos. 2005/0020503 A1 and 2005/0080005 A1, both herein incorporated by reference. Further examples of such anti-HCV agents are described in U.S. Patent Application Publication No. US 2005/0267151 A1, also incorporated by reference herein. The '151 publication also describes processes for the synthesis of substituted sulfonyl quinolines intermediates useful for preparing the agents. The substituted sulfonyl quinolines are prepared by amide coupling followed by cyclization in the presence of a strong base, tosylation and sulfonylation under acid conditions. However, there is a continuing need to develop alternative processes which may be more practical and economically useful for the preparation of these substituted sulfonyl quinolines.

Among the problems addressed by the present invention is the provision of a process that allows the use of economical reagents and requires a low number of operation steps for the manufacture of these compounds.

### SUMMARY OF THE INVENTION

The substituted sulfonyl quinolines of the present invention are prepared from substituted aromatic amino-ketones via amide formation with an acid followed by cyclization in the presence of an alkali or alkaline earth metal base and further conversion to a sulfone via a sulfonate intermediate. The present invention has the advantage of utilizing low cost, a lower number of steps and readily available starting materials and reagents. In addition, this procedure avoids the need for isolation of some intermediates, and minimizes the number of reagents operations for an overall faster cycle time.

One embodiment of the process of the present invention can be briefly summarized by the following scheme:

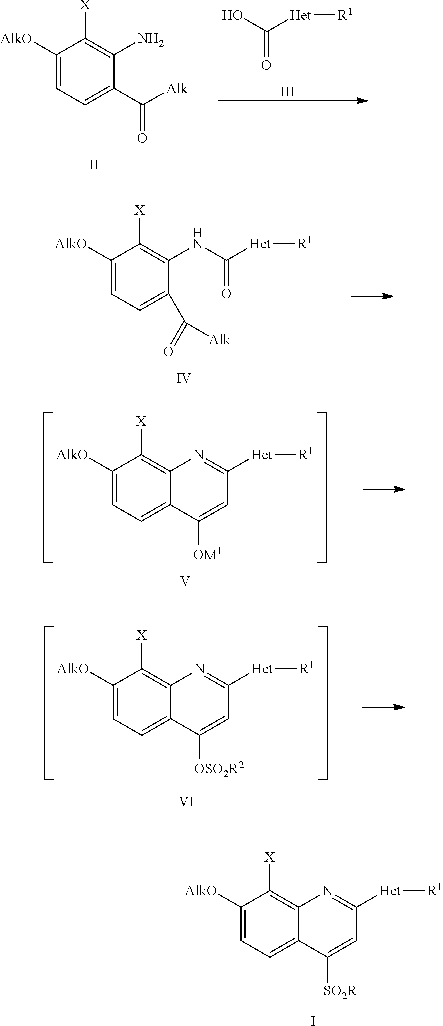

in which each Alk is independently C₁-C₆ alkyl; X is a halogen atom; R is C₁-C₁₀ alkyl, aryl, particularly C₆, or heteroaryl; R¹ is alkyl, particularly C₁-C₁₀ alkyl, more particularly C₁-C₆ alkyl, more particularly C₁-C₃ alkyl, or such alkyl which is optionally interrupted by one or more heteroatoms, e.g., —O—, —NH—, —C(═O)—, —N—(C₁-C₁₀ alkyl)-, —S—, or R¹ is (C₃₋₇)cycloalkyl and (C₃₋₇)cycloalkyl(C₁₋₄)alkyl-, wherein said cycloalkyl or cycloalkylalkyl may be mono-, di- or tri-substituted with (C₁₋₃)alkyl; M¹ is an alkali or alkali earth metal such as Na⁺, K⁺, Cs⁺, Mg²⁺ or Ca²⁺; R² is C₁-C₁₀ alkyl, aryl, particularly C₆, or heteroaryl, and Het is a heterocyclic radical as defined below. Each of the alkyl, aryl, heteroaryl and Het groups may optionally be substituted by alkyl, cycloalkyl, alkoxy, cycloalkoxy, phenylalkyl, alkenyl, amino, substituted amino, or amido (i.e., —NH—CO—R or —CO—NH—R).

Examples of the intermediate compounds of formula I which can be prepared according to the invention are described in U.S. Patent Application Publication No. 2005/0267151 A1, all incorporated by reference herein. Further examples of such compounds are as follows:

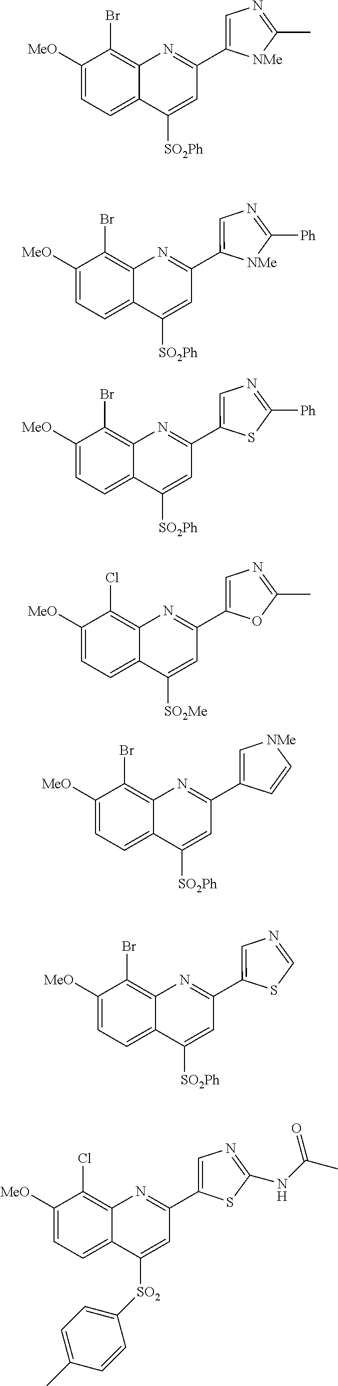

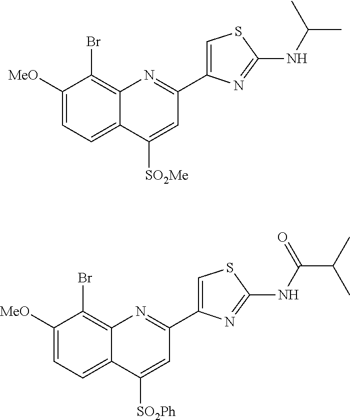

### DETAILED DESCRIPTION OF THE INVENTION

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined above and below, the number of carbon atoms is often specified preceding the group, for example, (C₁₋₁₀)alkyl means an alkyl group or radical having 1 to 10 carbon atoms and (C₃₋₇)cycloalkyl means a cycloalkyl group having from 3 to 7 carbon atoms in the ring. In general, for groups comprising two or more subgroups, the last named group is the point of attachment for the radical. For example, “cycloalkylalkyl” means a monovalent radical of the formula cycloalkyl-alkyl- and phenylalkyl means a monovalent radical of the formula phenyl-alkyl-. Unless otherwise specified below, conventional definitions of terms control and conventional stable atom valences are presumed and achieved in all formulas and groups.

The term “alkyl” as used herein, either alone or in combination with another substituent, means acyclic, straight or branched chain alkyl substituents containing the specified number of carbon atoms.

The term “alkoxy” as used herein, either alone or in combination with another substituent, means an alkyl group as defined above linked as a substituent through an oxygen atom: alkyl-O—.

The term “aryl” such as “C₆ or C₁₀ aryl” as used herein, either alone or in combination with another substituent, means either an aromatic cyclic system containing the stated number of carbon atoms, for example, an aromatic monocyclic system containing 6 carbon atoms or an aromatic bicyclic system containing 10 carbon atoms. For example, aryl includes a phenyl or a naphthyl ring system.

The term “Het” as used herein, either alone or in combination with another substituent, means a monovalent substituent derived by removal of a hydrogen from a five-, six-, or seven-membered saturated or unsaturated (including aromatic) heterocycle containing carbon atoms and from one to four ring heteroatoms selected from nitrogen, oxygen and sulfur. Examples of suitable heterocycles for providing the Het groups include: tetrahydrofuran, thiophene, diazepine, isoxazole, piperidine, dioxane, morpholine, pyrimidine or

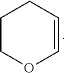

The term “Het” also includes those from a heterocycle as defined above fused to one or more other cyclic moiety, i.e., either a heterocycle or a carbocycle, each of which may be saturated or unsaturated. One such example includes thiazolo[4,5-b]-pyridine. Although generally included within the term “Het”, the term “heteroaryl” as used herein precisely defines an unsaturated heterocycle for which the double bonds form an aromatic system. Suitable examples of heteroaromatic systems include: quinoline, indole, pyridine,

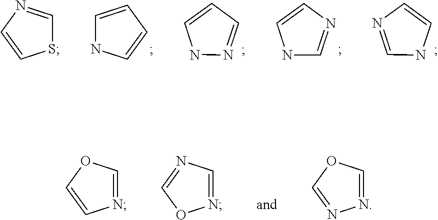

In general, all tautomeric forms and isomeric forms and mixtures, whether individual geometric isomers or optical isomers or racemic or non-racemic mixtures of isomers, of a chemical structure or compound are intended, unless the specific stereochemistry or isomeric form is specifically indicated in the compound name or structure.

The following chemicals may be referred to by these abbreviations:

| Abbreviation | Chemical Name |
|---|---|
| ACN | Acetonitrile |
| DMF | N,N-Dimethylformamide |
| DMSO | Dimethylsulfoxide |
| KDMO | Potassium 3,7-dimethyl-3-octanoxide |
| NMP | 1-Methyl-2-pyrrolidinone |
| THF | Tetrahydofuran |
| MeTHF | Methyltetrahydrofuran |
| DME | Dimethylether |

In the synthetic schemes below, unless specified otherwise, all the substituent groups in the chemical formulas shall have the same meanings as described herein unless otherwise specified. The reactants used in the synthetic schemes described below may be obtained either as described herein, or if not described herein, are themselves either commercially available or may be prepared from commercially available materials by methods known in the art. Certain starting materials, for example, may be obtained by methods described in U.S. Patent Application Publication No. US 2005/0267151 A1.

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography or High Pressure Liquid Chromatography (HPLC), if desired, and intermediates and products may be purified by chromatography on silica gel and/or by recrystallization, and characterized by one or more of the following techniques: NMR, mass spectroscopy and melting point.

In one embodiment, the present invention is directed to the following general multi-step synthetic method for preparing the compounds of formula I as set forth in Scheme I below. In other embodiments, the invention is directed to each of the individual steps of Scheme I and any combination of two or more successive steps of Scheme I. The invention may also be directed to the intermediate compounds set forth in Scheme I.

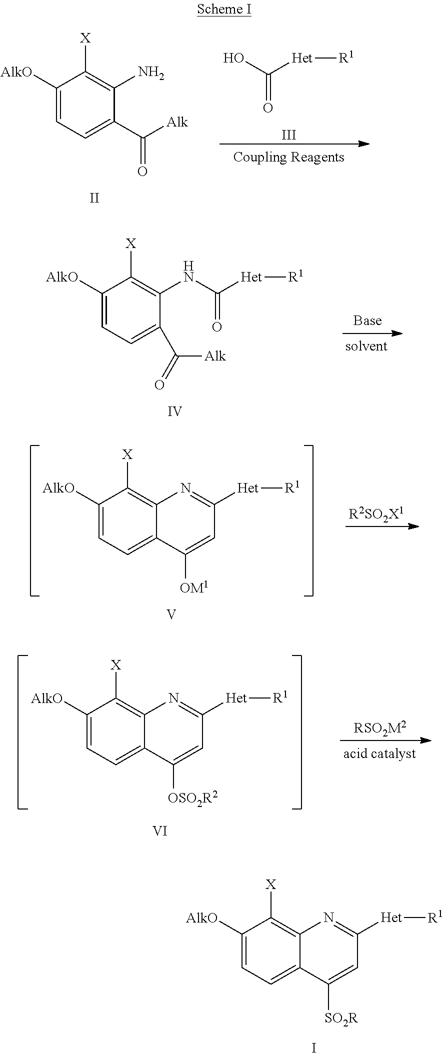

in which each Alk is independently C₁-C₆ alkyl; X and X¹ are independently halogen atoms; R is C₁-C₁₀ alkyl, C₆ aryl or heteroaryl; R¹ is alkyl, particularly C₁-C₁₀ alkyl, more particularly C₁-C₆ alkyl, more particularly C₁-C₃ alkyl, or such alkyl which is optionally interrupted by one or more heteroatoms, e.g., —O—, —NH—, —C(═O)—, —N—(C₁-C₁₀ alkyl)-, —S—, or R¹ is (C₃₋₇)cycloalkyl and (C₃₋₇)cycloalkyl(C₁₋₄)alkyl-, wherein said cycloalkyl or cycloalkylalkyl may be mono-, di- or tri-substituted with (C₁₋₃)alkyl; M¹ and M² are, independently, an alkali or alkali earth metal such as Na⁺, K⁺, Cs⁺, Mg²⁺ or Ca²⁺; R² is C₁-C₁₀ alkyl, C₆ aryl or heteroaryl, and Het is a heterocyclic radical as defined above.

In the first step, compound II is acylated with compound III to obtain compound IV. For the conversion of II to IV, acylation is achieved by either first converting carboxylic acid III to an activated form such as an acid chloride or by using standard peptide coupling protocols. The preferred method is to create the acid chloride of compound III using oxalyl chloride or thionyl chloride. This activated species is then coupled with the aniline compound II in any organic solvent or in water, with or without an added base. The preferred solvents are MeCN, NMP and THF and the preferred base (if used) is triethylamine. The reaction temperature is preferably from −30° C. to 150° C., more preferably from −20° C. to 50° C. Compound IV can be isolated, or alternatively be used for next step directly without isolation.

In the next steps, compound IV is cyclized in the presence of an alkali metal or alkaline earth metal base to obtain compound V as an alkali metal or alkaline earth metal salt. Compound V can be isolated and purified as its neutral form (hydroxyquinoline) by neutralization and filtration. But, preferably, it is subjected to sulfonylation conditions directly without isolation in a one-pot process to furnish sulfonate VI. The sulfonate VI is in turn converted to final compound I by reaction with a sulfonate salt. Preferably, the conversion from IV to I is also performed directly without isolation so that the three steps of proceeding from compound IV to compound I are performed all in a one-pot process.

For the conversion of IV to V in Scheme I, any alkali metal or alkaline earth metal base capable of forming the enolate can be used, for example, an alkali metal or alkaline earth metal hydroxide, such as KOH, NaOH, CaOH₂, and the like, with KOH being preferred. Any solvent which does not react with the enolate can be used, such as water, t-BuOH, THF, dioxane, DMSO, NMP, DME, mixtures thereof and the like, with water or a mixture of THF-water being preferred. The cyclization is preferably performed at a temperature of from 25° C. to 150° C., with 50° C. to 100° C. being particularly preferred.

For the conversion of V to VI in Scheme I, many sulfonylation reagents could be used, such as methanesulfonyl chloride, benzenesulfonyl chloride (PhSO₂Cl), toluenesulfonyl chloride (TsCl) and the like, with PhSO₂Cl and p-TsCl being preferred. The sulfonylation reaction may be carried out in the same (e.g., if included in a one-pot process) or a different solvent as used in previous step. Any solvent which does not react with the sulfonylation reagent may be used, such as water, DME, diglyme, THF, halocarbons, mixtures thereof, and the like, with THF-water or Me-THF-water mixture being preferred. The reaction temperature is preferably from −20° C. to 150° C. with 0-25° C. being particularly preferred.

For the conversion of VI to I in Scheme I, any sulfonate salt RSO₂M² can be used, where R is as defined previously and M² is an alkali or alkali earth metal, with PhSO₂Na, MeSO₂Na and p-MeC₆H₄SO₂Na being preferred. The reaction can be catalyzed by an acid such as HCl, MeSO₃H, H₂SO₄, p-TsOH, H₃PO₄, HOAc, HO₂H, CF₃CO₂H etc., with HCl being preferred. The sulfone formation step can be run in the same solvent (e.g., if included in a one-pot process) or a different solvent as used in previous step. Any solvent which does not react with the sulfonate VI may be used, such as water, DME, diglyme, THF, halocarbons and the like, with THF-water or a Me-THF-water mixture being preferred. The reaction temperature is preferably from −20° C. to 150° C. with 25-100° C. being particularly preferred.

In another embodiment, the invention is directed to a synthetic method which comprises the above-described step of cyclizing compound IV in the presence of an alkali metal hydroxide or alkaline earth metal hydroxide base to obtain compound V as an alkali metal or alkaline earth metal salt and, in a one-pot process without isolation or neutralization of compound V, subjecting compound V to sulfonylation directly to produce the sulfonate VI. Additionally, the invention is directed to a synthetic method comprising this step coupled with one or more of the other steps described above for Scheme I. For example, one embodiment of the invention is directed to the synthetic method of this step further comprising, in the same one-pot process without isolation of the sulfonate VI, converting to final compound I directly.

Preferred Alk, R, R¹, R², X, X¹, Het, M¹ and M² groups in the compounds of formulas II, III, IV, V, VI and I, include:

(A) Preferred definitions of Alk:
(i) C₁₋₆ alkyl,
(ii) methyl.
   (B) Preferred definitions of R:
(i) C₁₋₆ alkyl, C₆ aryl or heteroaryl,
(ii) phenyl or methyl.
   (C) Preferred definitions of X and X¹, independently:
(i) Cl, Br, or I,
(ii) Br.
   (D) Preferred definitions of Het:

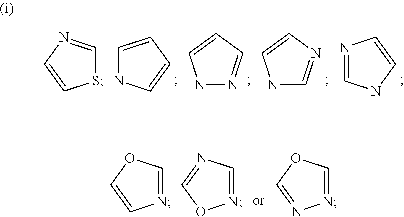

(ii) quinoline, indole, or pyridine;
(iii) tetrahydrofuran, thiophene, diazepine, isoxazole, piperidine, dioxane, morpholine, pyrimidine or

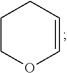

and
(iv) thiazolo[4,5-b]-pyridine.
   (E) Preferred definitions of M¹ and M²:
(i) M¹ is K,
(ii) M² is Na.
   (F) Preferred definitions of R¹:
(i) R¹ is R²⁰, —NR²²COR²⁰, —NR²²COOR²⁰—NR²²R²¹ and —NR²²CONR²¹R²³, wherein R²⁰ is selected from (C₁₋₈)alkyl, (C₃₋₇)cycloalkyl and (C₃₋₇)cycloalkyl(C₁₋₄)alkyl-, wherein said cycloalkyl or cycloalkylalkyl may be mono-, di- or tri-substituted with (C₁₋₃)alkyl; R²¹ is H or has one of the meanings of R²⁰ as defined above; and R²² and R²³ are independently selected from H and methyl. More preferably, R¹ is —NH—C(O)-Alk or —NH-Alk.
   (G) Preferred definitions of R²:
(i) C₁₋₆ alkyl, aryl or heteroaryl,
(ii) phenyl or methyl.
   Additional embodiments are wherein:
(i) any of the above groups are substituted with: alkyl, cycloalkyl, alkoxy, cycloalkoxy, phenylalkyl, alkenyl, amino, substituted amino, or amido (i.e., —NH—CO—R or —CO—NH—R).

In another embodiment, the present invention is directed to the intermediate compounds of formula V:

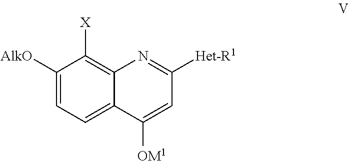

wherein Alk, X, M¹, Het and R¹ are as defined above. In a preferred embodiment of the compounds of formula V: X is halo, particularly bromine, Alk is methyl and Het-R¹ is thiazole substituted by a —NH—C(O)—C₁-C₆ alkyl or —NH—C₁-C₆ alkyl group.

Applicants have discovered that the cyclization to obtain the quinoline compound V using an alkali metal or alkaline earth metal base is advantageous since the use of a strong base, such as t-BuOK, KDMO or lithium diisopropylamide, can be avoided. Thus, the later step of quenching the base with an acid is made easier. Further, provision of the alkali metal or alkaline earth metal salt compound V facilitates the sulfonylation reaction, without isolation. This salt reacts better than the neutralized hydroxyquinoline and it is not necessary to add additional base to conduct the reaction. Thus, there is a lower material requirement, less steps and a more environmentally benign result is achieved. In a further embodiment, a further advantage is obtained in that the method allows for a solvent comprising water in the cyclization step to compound V and the other steps conducted in one-pot with that step.

Specific embodiments of the invention are further described by the following non-limiting synthetic examples and description of specific embodiments.

### Synthetic Examples

### Example 1

### Synthesis

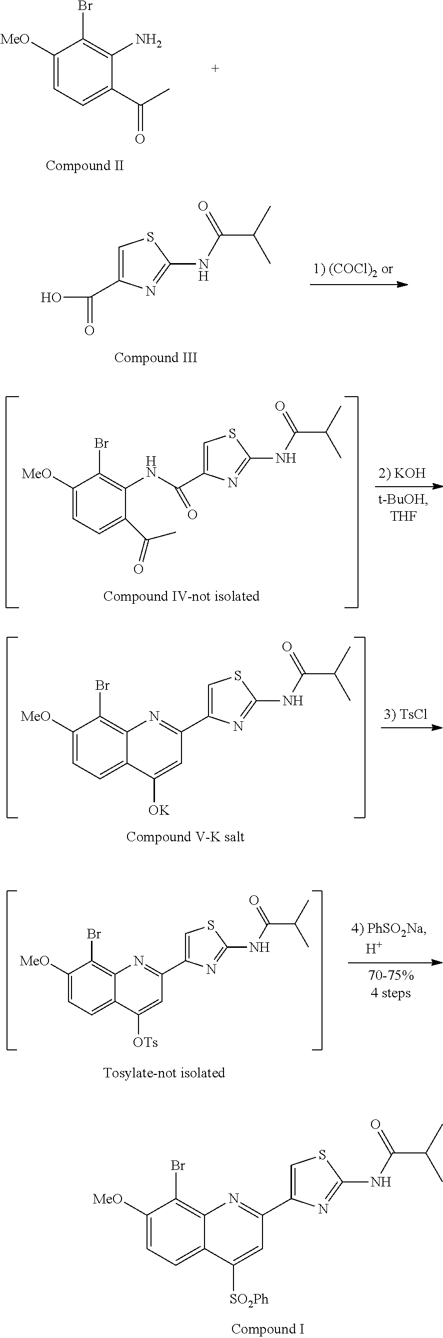 [0047] 1. Charge the thiazole compound III and NMP to a reactor.
2. Charge thionyl chloride after 15 min., keeping the temperature below 25° C.
3. Stir batch at 25° C. for 0.5 h. Check HPLC with PrNH₂ to confirm formation of acid chloride is complete (2 drops sample is added to 1 ml MeCN+0.1 ml PrNH₂, Rt=5.05 min for propyl amide, Rt=4.16 min for remaining acid, target <1%).
4. Charge solution of the aniline compound II in MeTHF at 25° C. and stir for 12 h at 30° C. until HPLC shows <2% of the aniline compound.
5. Charge 15% NaOH solution slowly keeping inside temperature below 22° C. Quench is exothermic. Set the jacket temperature at 0° C. The pH of the aqueous layer is measured to about 7.
6. Charge MeTHF and then add water and stir for 5 min and then allow the layers to separate at 40° C.
7. Wash with 5 wt % NaHCO₃ and brine and separate the layers at 22° C.
8. Distill MeTHF and switch solvent to THF to adjust the final volume to about 310 ml.
9. Charge t-BuOH and heat the contents to internal temperature 65° C.
10. Charge 50 wt % KOH solution at 65° C. and stir for 12-14 h until HPLC shows Compound IV is <1%.
11. Charge benzenesulfonyl chloride (TsCl) at 10° C. over minimum of 1 h and then stir at 22° C. for 0.5 h.
12. Charge suspension of benzenesulphinic acid Na salt in water at 22° C. followed by 2M HCl and stir at 54-56° C. for 12-14 h until HPLC shows tosylate is <1%.
13. Cool to 22° C. and charge 5 wt % NaHCO₃ and brine and separate the layers.
14. Distill THF and switch solvent to DMF and then charge water at 50° C. over 30 min and slowly cool the batch to 22° C. over 2 h.
15. Charge 1M NaOH and stir for 30 min at 22° C.
16. Filter the slurry, rinse with 1.5:1 DMF/water and dry under vacuum at 50° C. for 12-15 h to afford 32.4 g of solid purple solid of compound I (as mono solvate of DMF) (70% isolated yield).

### Example 2

### Synthesis

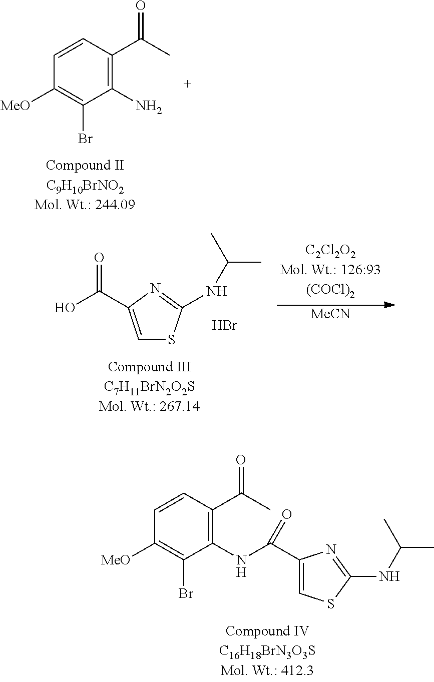

| Material | MW | Amount | Mol | Eq |
|---|---|---|---|---|
| Compound III | 267 | 4.005 Kg | 15 | 1.0 |
| Compound II | 244 | 3.660 g | 15 | 1.0 |
| MeCN | | 40 L | | |
| DMF | 73 | 160 g | | 0.04 vol |
| Oxallyl chloride | 126.9 | 2.0 Kg | 15.75 | 1.05 |
| Et₃N | 101 | 1.63 Kg + 3.0 Kg | 16.1 | 1.076 |

1. Add Compound III. Add MeCN. Add DMF. Cool the batch to 10° C.
2. Add oxalyl chloride after 15 min., keeping temperature at 10-15° C. and gas bubbling under control. Addition time could be longer in larger scale if it is necessary for control of bubbling. Stir batch at 27° C. for 5 h. A clear solution is obtained. Check HPLC with PrNH₂ to confirm formation of acid chloride is complete (2 drops sample is added to 1 ml MeCN+0.1 ml PrNH₂, Rt=4.3 min for propyl amide, Rt=2.6 min for remaining acid, target <1%).
3. Add solid Compound II at 0-5° C., stir for 1 h at 10° C.
4. Add Et₃N at 10-13° C. in 40-60 min., stir at 13° C. for 6 h and 23° C. for overnight (11 h). Check HPLC to make sure Compound II (Rt=7.9 min) is <1% area. It is recommended to apply agitation at fast speed several times in the middle of this period for good mixing effect.
5. Cool to 10° C.
6. Add 20 l water, keeping inside temperature below 2° C. Exotherm is very minor
7. Add about 4.0 l Et₃N, keeping inside temperature at 20-25° C. Note mild exotherm, expect temperature rising about 5° C. Stir slurry at 22° C. for 1 h. Check pH is 7-8.
8. Filter slurry, and rinse with 18 l acetonitrile-water (1:2).
9. Compound IV Yield: 75%. 4.56 Kg.

### Example 3

### Synthesis

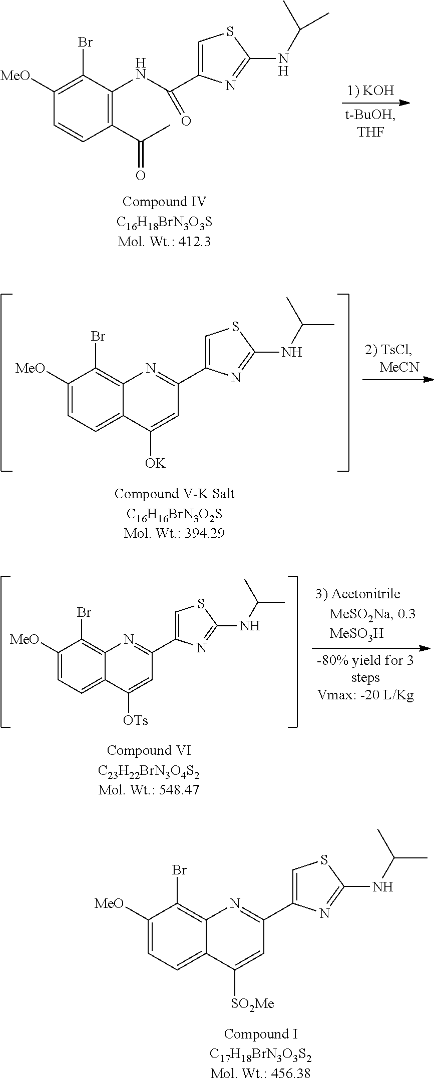

| Starting Material | MW | Amount | Mol | Eq. |
|---|---|---|---|---|
| Compound IV | 412.303 | 4.12 Kg | 10 | 1.0 |
| KOH | 56.11 | 645 g | 11 | 1.15 |
| t-BuOH (d = 0.775) | 74.12 | 16 L | — | Solvent |
| THF (d = 0.889) | 72.11 | 28 L | — | Solvent |
| TsCl | 190.648 | 1.906 g | 10 | 1.0 |
| MeCN (d = 0.867) | 90.12 | 28 L | — | Solvent |
| Methanesulphinic | 102.089 | 1.53 g | 15 | 1.5 |
| acid Na salt | | | | |
| Methanesulfonic acid | 96.10 | 288 g | 3 | 0.3 |
| (d = 1.48) | | | | |
| NaHCO₃ | 105.9 | 790 g | 7.5 | 0.75 |
| DI water | 18.00 | 34 L | — | — |

1. Charge Compound IV and solid KOH (505 g, 0.9 eq) into 50 L reactor.
2. Charge THF (28 L) and t-BuOH (16 L) into the reactor.
3. Raise the batch temperature to 65° C. and stir for 2.0 h.
4. Check by HPLC.
5. Add remaining KOH (140 g, 0.25 eq) and stir for about 10 h at 65-67° C. until HPLC shows Compound IV is <1.0%
6. Cool to 5-10° C. and charge tosyl chloride solution (1.906 Kg of TsCl in 4 L MeCN) over 1 h while keeping the internal temperature at about 10° C.
7. Distill solvents at 45° C. under vacuum to about 20 L, then add MeCN (28 L) and cool to 30° C.
8. Add methanesulphinic acid Na salt (1.530 Kg, 1.5 eq) at 30° C. and then methanesulfonic acid (288 g, 0.3 eq) keeping agitation in fast speed. Slight exotherm observed (˜1° C.).
9. Heat the contents to 50° C. and stir for 6 h until HPLC shows Compound VI is <0.5%. Reaction can be run overnight without further decomposition.
10. Upon completion, cool to 25° C. Add aqueous NaHCO₃ solution (0.75 eq, 790 g in 24 L of water) in 15 min to adjust the pH to 7.
11. Stir for 1 h at 22° C.
12. Filter, and rinse with 1:1 mixture of ACN:Water (12 L) and then with water (10 L).
13. Dry the solid at 60° C. under reduced pressure with a bleed of N₂ until KF is <0.2%. Yield 3.5 Kg (˜78%) of Compound I as a green solid.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

### Specific Embodiments

A. A method comprising reacting a compound of formula IV with an alkali metal or alkaline earth metal base in the presence of a solvent to obtain an alkali metal or alkaline earth metal salt compound of formula V:

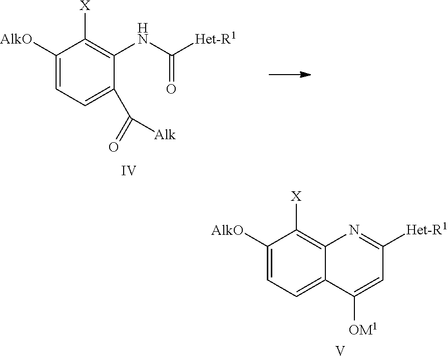

wherein Alk is a C₁-C₆ alkyl group; X is a halogen atom; M¹ is an alkali metal or alkali earth metal; R¹ is alkyl, particularly C₁-C₁₀ alkyl, more particularly C₁-C₆ alkyl, more particularly C₁-C₃ alkyl, or such alkyl which is optionally interrupted by one or more heteroatoms, e.g., —O—, —NH—, —C(═O)—, —N—(C₁-C₁₀ alkyl)- or —S—, or R¹ is (C₃₋₇)cycloalkyl and (C₃₋₇)cycloalkyl(C₁₋₄)alkyl-, wherein said cycloalkyl or cycloalkylalkyl may be mono-, di- or tri-substituted with (C₁₋₃)alkyl; and Het is a monovalent substituent derived by removal of a hydrogen from a five-, six-, or seven-membered saturated or unsaturated (including aromatic) heterocycle containing carbon atoms and from one to four ring heteroatoms selected from nitrogen, oxygen and sulfur; wherein each of the alkyl, aryl, heteroaryl and Het groups above are optionally independently substituted by alkyl, cycloalkyl, alkoxy, cycloalkoxy, amino, amido or aryl.

B. A method comprising:
reacting a compound of formula IV with an alkali metal or alkaline earth metal base in the presence of a solvent to obtain an alkali metal or alkaline earth metal salt compound of formula V, and
without isolating the compound of formula V, further reacting the resulting product with a sulfonylation reactant to obtain a compound of the formula VI:

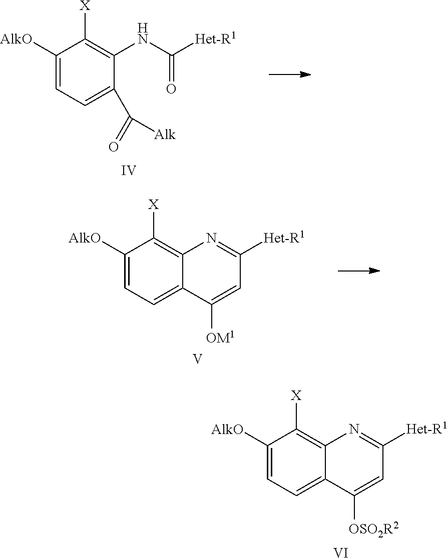

wherein Alk, X, M¹, R¹ and Het are as defined for method A. above and R² is C₁-C₁₀ alkyl, aryl, preferably C₆, or heteroaryl; wherein each of the alkyl, aryl, heteroaryl and Het groups above are optionally independently substituted by alkyl, cycloalkyl, alkoxy, cycloalkoxy, phenylalkyl, alkenyl, amino, substituted amino, or amido (i.e., —NH—CO—R or —CO—NH—R, wherein R is C₁-C₁₀ alkyl).

C. The method B. described above further comprising reacting compound VI with a sulfonate salt RSO₂M² to obtain a compound of the formula I:

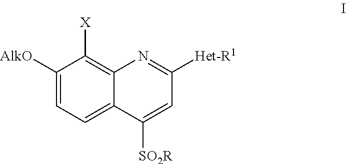

wherein Alk, X, M¹, R¹, R² and Het are as defined above; R is C₁-C₁₀ alkyl, aryl, preferably C₆, or heteroaryl; and M² is an alkali or alkali earth metal; wherein each of the alkyl, aryl, heteroaryl and Het groups above are optionally independently substituted by alkyl, cycloalkyl, alkoxy, cycloalkoxy, phenylalkyl, alkenyl, amino, substituted amino, or amido (i.e., —NH—CO—R or —CO—NH—R, wherein R is C₁-C₁₀ alkyl).

D. The above method C. wherein the compound VI is not isolated before reacting with the sulfonate salt.

E. Any of the above methods A., B., C. or D., which further comprises obtaining compound IV by acylating compound II with compound III in the presence of a solvent, and optionally with addition of a base, to obtain compound IV, the acylation being achieved by either first converting compound III to an acid chloride activated form or by using peptide coupling methods:

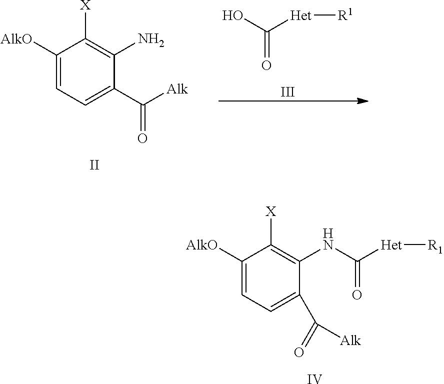

wherein Alk, X, R¹ and Het are as defined above, each Alk being independently selected.

F. Any of the above methods A., B., C., D. or E., wherein the alkali metal or alkaline earth metal base is an alkali metal or alkaline earth metal hydroxide.

G. Any of the above methods A., B., C., D. or E., wherein the alkali metal or alkaline earth metal base is a potassium hydroxide.

H. Any of the above methods E., F. or G., wherein compound II is converted to an acid chloride by reaction with oxalyl chloride or thionyl chloride.

I. Any of the above methods E., F., G. or H., where the solvent for the acylation of compound II with compound III comprises MeCN, NMP or THF and the optional base is triethylamine and the reaction is conducted at a temperature of from −30° C. to 150° C.

J. Any of the above methods A., B., C., D., E., F., G., H. or I., wherein the solvent for the cyclization of compound IV comprises: water, t-BuOH, THF's, dioxane, DMSO, NMP, or DME and the cyclization reaction is performed at a temperature of from 25° C. to 150° C.

K. Any of the above methods A., B., C., D., E., F., G., H., or I., wherein the solvent for the cyclization of compound IV comprises water.

L. Any of the above methods B., C., D., E., F., G., H., I., J. or K., wherein the sulfonylation reagent for the conversion of compound V to compound VI is methanesulfonyl chloride, benzenesulfonyl chloride or toluenesulfonyl chloride and the reaction temperature for the conversion is from −20° C. to 150° C.

M. Any of the above methods C., D., E., F., G., H., I., J., K. or L., wherein the sulfonate salt RSO₂M² for the conversion of compound VI to compound I is PhSO₂Na, MeSO₂Na or p-MeC₆H₄SO₂Na.

N. Any of the above methods C., D., E., F., G., H., I., J., K., L. or M., wherein the conversion of compound VI to compound I is catalyzed by an acid selected from HCl, MeSO₃H, H₂SO₄, p-TsOH, H₃PO₄, HOAc, HO₂H, and CF₃CO₂H.

O. Any of the above methods C., D., E., F., G., H., I., J., K., L. or M., wherein the conversion of compound VI to compound I is catalyzed by HCl.

P. Any of the above methods C., D., E., F., G., H., I., J., K., L., M., N. or O., wherein the conversion of compound VI to compound I is at a reaction temperature of from −20° C. to 150° C.

Q. Any of the above methods A., B., C., D., E., F., G., H., I., J., K., L., M., N., O., or P., wherein, in the compounds:
Alk is methyl;
R is phenyl or methyl;
X is Cl, Br, or I;
Het is:

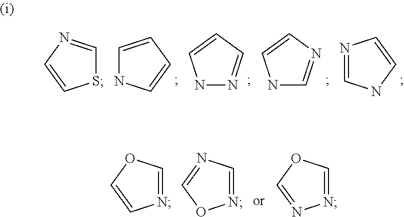

(iii) quinoline, indole, or pyridine;
(iii) tetrahydrofuran, thiophene, diazepine, isoxazole, piperidine, dioxane, morpholine, pyrimidine or

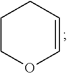

or
   (iv) thiazolo[4,5-b]-pyridine;
M¹ is K and M² is Na;
R¹ is R¹ is R²⁰, —NR²²COR²⁰, —NR²²COOR²⁰—NR²²R²¹ and —NR²²CONR²¹R²³, wherein R²⁰ is selected from (C₁₋₈)alkyl, (C₃₋₇)cycloalkyl and (C₃₋₇)cycloalkyl(C₁₋₄)alkyl-, wherein said cycloalkyl or cycloalkylalkyl may be mono-, di- or tri-substituted with (C₁₋₃)alkyl; R²¹ is H or has one of the meanings of R²⁰ as defined above; and R²² and R²³ are independently selected from H and methyl, most preferably, R¹ is —NH—C(O)-Alk or —NH-Alk; and
R² is phenyl or methyl.

R. An intermediate compound of formula V:

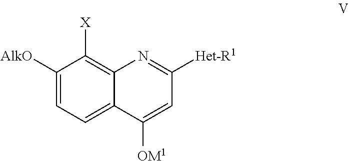

wherein Alk is a C₁-C₆ alkyl group; X is a halogen atom; M¹ is an alkali metal or alkali earth metal; R¹ is alkyl, particularly C₁-C₁₀ alkyl, more particularly C₁-C₆ alkyl, more particularly C₁-C₃ alkyl, or such alkyl which is optionally interrupted by one or more heteroatoms, e.g., —O—, —NH—, —C(═O)—, —N—(C₁-C₁₀ alkyl)- or —S—, or R¹ is (C₃₋₇)cycloalkyl and (C₃₋₇)cycloalkyl(C₁₋₄)alkyl-, wherein said cycloalkyl or cycloalkylalkyl may be mono-, di- or tri-substituted with (C₁₋₃)alkyl; and Het is a monovalent substituent derived by removal of a hydrogen from a five-, six-, or seven-membered saturated or unsaturated (including aromatic) heterocycle containing carbon atoms and from one to four ring heteroatoms selected from nitrogen, oxygen and sulfur; wherein each of the alkyl, aryl, heteroaryl and Het groups above are optionally independently substituted by alkyl, cycloalkyl, alkoxy, cycloalkoxy, phenylalkyl, alkenyl, amino, substituted amino, or amido (i.e., —NH—CO—R or —CO—NH—R).

S. An intermediate compound of embodiment R where: X is bromine, Alk is methyl and Het-R¹ is thiazole substituted by a —NH—C(O)—C₁-C₆ alkyl or —NH—C₁-C₆ alkyl group.

1. A method comprising reacting a compound of formula IV with an alkali metal or alkaline earth metal base in the presence of a solvent to obtain an alkali metal or alkaline earth metal salt compound of formula V: 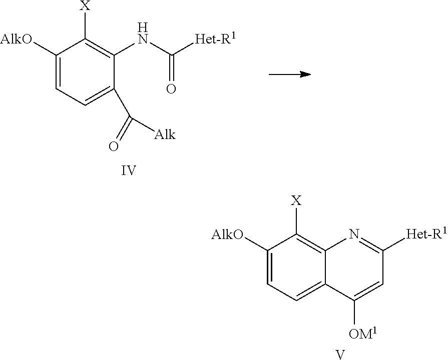
wherein each Alk is independently a C₁-C₆ alkyl group; X is a halogen atom; M¹ is an alkali metal or alkali earth metal; R¹ is C₁-C₁₀ alkyl, optionally interrupted by one or more of: —O—, —NH—, —C(═O)—, —N—(C₁-C₁₀ alkyl)- or —S—, or R¹ is (C₃-C₇)cycloalkyl or (C₃₋₇)cycloalkyl(C₁₋₄)alkyl-, wherein said cycloalkyl or cycloalkylalkyl may be mono-, di- or tri-substituted with (C₁₋₃)alkyl; and Het is a monovalent substituent derived by removal of a hydrogen from a five-, six-, or seven-membered saturated or unsaturated (including aromatic) heterocycle containing carbon atoms and from one to four ring heteroatoms selected from nitrogen, oxygen and sulfur; wherein each of the alkyl, aryl, heteroaryl and Het groups above are optionally independently substituted by alkyl, cycloalkyl, alkoxy, cycloalkoxy, amino, amido or aryl.

2. A method according to claim 1 comprising reacting a compound of formula IV with an alkali metal or alkaline earth metal base in the presence of a solvent to obtain an alkali metal or alkaline earth metal salt compound of formula V, and
without isolating the compound of formula V, further reacting the resulting product with a sulfonylation reactant to obtain a compound of the formula VI: 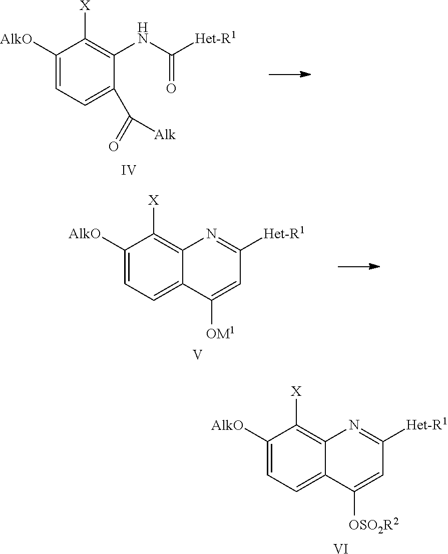
wherein Alk, X, M¹, R¹ and Het are as defined for method A above and R² is C₁-C₁₀ alkyl, aryl or heteroaryl; wherein each of the alkyl, aryl, heteroaryl and Het groups above are optionally independently substituted by alkyl, cycloalkyl, alkoxy, cycloalkoxy, phenylalkyl, alkenyl, amino, substituted amino or amido.

3. A method according to claim 2, further comprising reacting compound VI with a sulfonate salt RSO₂M² to obtain a compound of the formula I: 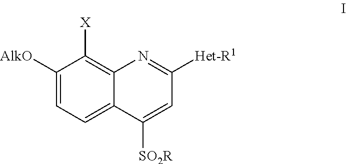
wherein Alk, X, R¹ and Het are as defined above; R is C₁-C₁₀ alkyl, aryl, or heteroaryl; and M² is an alkali or alkali earth metal; wherein each of the alkyl, aryl, heteroaryl and Het groups above are optionally independently substituted by alkyl, cycloalkyl, alkoxy, cycloalkoxy, phenylalkyl, alkenyl, amino, substituted amino, or amido.

4. A method according to claim 3 wherein the compound VI is not isolated before reacting with the sulfonate salt.

5. A method according to claim 1 which further comprises obtaining compound IV by acylating compound II with compound III in the presence of a solvent, and optionally with addition of a base, to obtain compound IV, the acylation being achieved by either first converting compound III to an acid chloride activated form or by using peptide coupling methods: 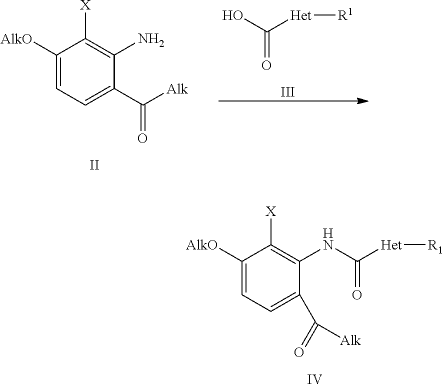
wherein Alk, X, R¹ and Het are as defined in claim 1, each Alk being independently selected.

6. A method according to claim 1, wherein the alkali metal or alkaline earth metal base is an alkali metal or alkaline earth metal hydroxide.

7. A method according to claim 1, wherein the solvent for the cyclization of compound IV comprises: water, t-BuOH, THF, dioxane, DMSO, NMP, or DME and the cyclization reaction is performed at a temperature of from 25° C. to 150° C.

8. A method according to claim 1, wherein the solvent for the cyclization of compound IV comprises water.

9. A method according to claim 2, wherein the sulfonylation reagent for the conversion of compound V to compound VI is methanesulfonyl chloride, benzenesulfonyl chloride or toluenesulfonyl chloride and the reaction temperature for the conversion is from −20° C. to 150° C.

10. A method according to claim 3, wherein the sulfonate salt RSO₂M² for the conversion of compound VI to compound I is PhSO₂Na, MeSO₂Na or p-MeC₆H₄SO₂Na.

11. A method according to claim 3, wherein the conversion of compound VI to compound I is catalyzed by an acid selected from HCl, MeSO₃H, H₂SO₄, p-TsOH, H₃PO₄, HOAc, HO₂H, and CF₃CO₂H.

12. A method according to claim 3, wherein the conversion of compound VI to compound I is catalyzed by HCl.

13. A method according to claim 3, wherein the conversion of compound VI to compound I is at a reaction temperature of from −20° C. to 150° C.

14. An intermediate compound of formula V: 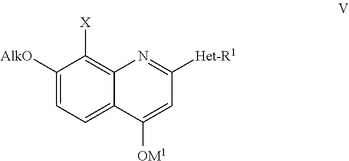
wherein Alk is a C₁-C₆ alkyl group; X is a halogen atom; M¹ is an alkali metal or alkali earth metal; R¹ is C₁-C₁₀ alkyl, optionally interrupted by one or more of: —O—, —NH—, —C(═O)—, —N—(C₁-C₁₀ alkyl)- or —S—, or R¹ is (C₃₋₇)cycloalkyl or (C₃₋₇)cycloalkyl(C₁₋₄)alkyl-, wherein said cycloalkyl or cycloalkylalkyl may be mono-, di- or tri-substituted with (C₁₋₃)alkyl; and Het is a monovalent substituent derived by removal of a hydrogen from a five-, six-, or seven-membered saturated or unsaturated (including aromatic) heterocycle containing carbon atoms and from one to four ring heteroatoms selected from nitrogen, oxygen and sulfur; wherein each of the alkyl, aryl, heteroaryl and Het groups above are optionally independently substituted by alkyl, cycloalkyl, alkoxy, cycloalkoxy, phenylalkyl, alkenyl, amino, substituted amino, or amido.

15. An intermediate compound of formula V according to claim 14, wherein: X is bromine, Alk is methyl and Het-R¹ is thiazole substituted by a —NH—C(O)—C₁-C₆ alkyl or —NH—C₁-C₆ alkyl group.
